(19) United States
(12) Patent Application Publication
(10) Pub. No.: US 2021/0196428 A1
(43) Pub. Date: Jul. 1, 2021

(54) Artificial Intelligence (AI) based Decision-Making Model for Orthodontic Diagnosis and Treatment Planning

(71) Applicant: University of Connecticut, Farmington, CT (US)

(72) Inventors: Madhur Upadhyay, Newington, CT (US); Yasir Suhail, Farmington, CT (US); Kshitz Gupta, W. Hartford, CT (US); Aditya Chhibber, Avon, OH (US)

(21) Appl. No.: 17/107,193

(22) Filed: Nov. 30, 2020

Related U.S. Application Data
(60) Provisional application No. 62/955,212, filed on Dec. 30, 2019.

(51) Int. Cl. A61C 7/00; G16H 50/20; G06N 5/00

(52) U.S. Cl. CPC A61C 7/002; G06N 5/003; G16H 50/20

(57) ABSTRACT

A computer-implemented method and corresponding system provide orthodontic treatment options for use in orthodontic diagnosis or treatment planning. The method performs a rules-based expert system analysis on a given feature variable to produce expert system treatment options. The given feature variable represents an orthodontic feature of a patient. The method applies a computer-implemented multi-component model to a given set of feature variables to produce multi-component model-based treatment options that include primary and secondary model-based treatment options. The method compares the expert system treatment options to the multi-component model-based treatment options to determine disagreement or agreement between each other, enabling a suitable treatment decision to be arrived at which can be valuable to clinicians for verifying treatment plans, minimizing human error, training orthodontists, and improving reliability.

### RELATED APPLICATION

This application claims the benefit of U.S. Provisional Application No. 62/955,212, filed on Dec. 30, 2019. The entire teachings of the above application are incorporated herein by reference.

### BACKGROUND

Extraction of teeth is an important treatment decision in orthodontic practice. Whether to perform extraction is often a controversial decision in orthodontic treatment because extractions are irreversible. Such decisions are based on clinical evaluations, patient photographs, dental study models, radiographs, and substantially rely upon the experience and knowledge of a clinician.

### SUMMARY

According to an example embodiment, a computer-implemented method for providing orthodontic treatment options for use in orthodontic diagnosis or treatment planning comprises performing a rules-based expert system analysis on a given feature variable to produce expert system treatment options. The given feature variable represents an orthodontic feature of a patient. The expert system treatment options are fewer in number than a number of standard orthodontic treatment options that apply to orthodontic diagnosis, orthodontic treatment options, or both. The method further comprises applying a computer-implemented multi-component model to a given set of feature variables to produce multi-component model-based treatment options that include primary and secondary model-based treatment options and comparing the expert system treatment options to the multi-component model-based treatment options to determine disagreement or agreement between each other. If disagreement, the method further comprises enabling an expert to review the expert system treatment options and the multi-component model-based treatment options, and adapting at least one of the given feature variable, rules-based expert system analysis, or multi-component model based on feedback from the expert. If agreement, the method further comprises outputting the primary and secondary model-based treatment options to a clinician.

The computer-implemented method may further comprise providing text-based information to the clinician with the primary and secondary model-based treatment options. The text-based information may relate to an interpretation produced by the multi-component model.

The multi-component model may include at least two computer-implemented methods that produce respective results having a characteristic of at least one of interpretability, reliability, or accuracy, wherein a result with at least one of each characteristic may be produced by the multi-component model.

The multi-component model may perform a multi-class logistic regression that produces a reliable result, interpretable result for a specific treatment option, or both. The specific treatment option may include a location or identity of tooth extraction or other multi-class diagnosis or treatment. The multi-component model may further perform a logistic regression that produces an interpretable and reliable result for an extraction option, non-extraction option, or other binary decision for diagnosis or treatment. The multi-component model may further perform a random forest method that produces an accurate result based on previous expert-decisions based training.

The multi-class logistic regression may be performed by a neural network including 0 or more hidden layers. The logistic regression, multi-class logistic regression, or combination thereof, may be replaced by a decision tree, linear regression, generalized linear model, decision rules, RuleFit, naïve Bayes, k nearest neighbors, or one or more other interpretable machine learning method. The random forest may be replaced by one or more of other machine learning methods with learning capacity and generalizability, which may or may not have interpretability, the one or more other machine learning methods including deep neural networks or other ensemble methods, the other ensemble methods including XGBoost, bagging, boosting, support vector machines, or a combination thereof.

The computer-implemented method may further comprise integrating goals of interpretability, reliability, and accuracy using one integrated machine learning method that fuses the ideas or components of other methods. The given feature variable may be a set of feature variables of a patient's orthodontia discernible from at least one of an x-ray, picture, physical model of the patient's orthodontia, or combination thereof. A number of feature variables in the set may be within a range of: 1-7, 1-70, or 1-700.

The computer-implemented method may further comprise performing automatic or user assisted feature identification on the x-ray, picture, model, or combination thereof, to produce the set of feature variables.

The given feature variable may be a set of feature variables and the method may further comprise (i) performing a corresponding rules-based expert system analysis on each feature variable of the set, (ii) applying the multi-component model to each feature variable, and (iii) performing the comparing, enabling, and outputting based on results of (i) and (ii).

The expert may be an expert clinician, expert panel of clinicians, or computer-implemented artificial intelligence or adaptive learning system.

The computer-implemented method may further comprise performing a safety check of the rules-based expert system analysis based on a result of the computer-implemented multi-component model and replacing the safety check by other well-accepted orthodontic standards.

Features used for the rules-based expert system analysis or computer-implemented multi-component model may be qualitative and categorical variables that are easily understood and used in the clinical setting.

The computer implemented method may further comprise enabling a user to interact with a central server implementing the computer-implemented multi-component model through use of a visual or text based interface on a computer, phone, tablet, or other electronic device.

The computer implemented method may further comprise enabling a user to select an option to store patient data of the patient either on selected equipment or on a central server.

The computer-implemented method may further comprise automatically deriving at least one orthodontic feature of the patient from patient X-rays or other images by human intervention.

The computer-implemented method may further comprise recommending or ruling out braces, aligners, tooth extraction, or other diagnoses or treatments.

According to another example embodiment, a system for providing orthodontic treatment options for use in orthodontic diagnosis or treatment planning comprises at least one processor configured to perform a rules-based expert system analysis on a given feature variable to produce expert system treatment options. The given feature variable represents an orthodontic feature of a patient. The expert system treatment options are fewer in number than a number of standard orthodontic treatment options that apply to orthodontic diagnosis, orthodontic treatment options, or both. The at least one processor is further configured to apply a computer-implemented multi-component model to a given set of feature variables to produce multi-component model-based treatment options that include primary and secondary model-based treatment options and compare the expert system treatment options to the multi-component model-based treatment options to determine disagreement or agreement between each other. If disagreement, the at least one processor is further configured to enable an expert to review the expert system treatment options and the multi-component model-based treatment options, and adapting at least one of the given feature variable, rules-based expert system analysis, or multi-component model based on feedback from the expert. If agreement, the at least one processor is further configured to output the primary and secondary model-based treatment options to a clinician.

The system may be integrated into an electronic medical records system.

Alternative system embodiments parallel those described above in connection with the example method embodiment.

According to another example embodiment, a non-transitory computer-readable medium for providing orthodontic treatment options for use in orthodontic diagnosis or treatment planning has encoded thereon a sequence of instructions which, when loaded and executed by at least one processor, causes the at least one processor to perform a rules-based expert system analysis on a given feature variable to produce expert system treatment options. The given feature variable represents an orthodontic feature of a patient. The expert system treatment options are fewer in number than a number of standard orthodontic treatment options that apply to orthodontic diagnosis, orthodontic treatment options, or both. The sequence of instructions further causes the at least one processor to apply a computer-implemented multi-component model to a given set of feature variables to produce multi-component model-based treatment options that include primary and secondary model-based treatment options and compare the expert system treatment options to the multi-component model-based treatment options to determine disagreement or agreement between each other. If disagreement, the sequence of instructions further causes the at least one processor to enable an expert to review the expert system treatment options and the multi-component model-based treatment options, and adapting at least one of the given feature variable, rules-based expert system analysis, or multi-component model based on feedback from the expert. If agreement, the sequence of instructions further causes the at least one processor to output the primary and secondary model-based treatment options to a clinician.

Alternative non-transitory computer-readable medium embodiments parallel those described above in connection with the example method embodiment.

It should be understood that example embodiments disclosed herein can be implemented in the form of a method, apparatus, system, or computer readable medium with program codes embodied thereon.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments, as illustrated in the accompanying drawings in which like reference characters refer to the same parts throughout the different views. The drawings are not necessarily to scale, emphasis instead being placed upon illustrating embodiments.

FIG. 1A is a block diagram of an example embodiment of a system that provides orthodontic treatment options for use in orthodontic diagnosis or treatment planning.

FIG. 1B is an outline of a workflow that simulates an expert's decision of whether teeth need to be 1) extracted or 2) not extracted.

FIG. 2 is patient record that includes a set of feature variables 210 that may be employed as the clinical variables of FIG. 1B.

FIG. 3 is a decision diagram of an example embodiment of decisions of a model for determining a treatment option

FIG. 4 is an image of an example embodiment of teeth.

FIG. 5 is a block diagram of example embodiments of methods for providing orthodontic treatment options for use in orthodontic diagnosis or treatment planning.

FIG. 6 is a flow diagram of an example embodiment of a computer-implemented method for providing orthodontic treatment options for use in orthodontic diagnosis or treatment planning.

FIG. 7 is a block diagram of an example embodiment of a workflow from data collection to the machine learning diagnosis.

FIG. 8A is a graph of an example embodiment of the age distribution of patients from whom patient data was taken for a study.

FIG. 8B is a graph of an example embodiment of the gender distribution of the patients from whom the patient data was taken for the study.

FIG. 9A is a graph that shows performance of a logistic regression model and a multinomial regression neural network model when considering only a primary diagnosis.

FIG. 9B is a graph that shows performance of a logistic regression model and a multinomial regression neural network model when considering both the primary and alternative diagnosis.

FIG. 10 is a graph an example embodiment of training time for single classifiers.

FIGS. 11A and 11B are graphs of an example embodiment of an effect of various training parameters on the Random Forest model for the prediction of the specific extraction.

FIGS. 12A and 12B are graphs of an example embodiment of an effect of various training parameters on the Random Forest model for the binary predication problem.

FIG. 13 is graph of a saturating effect of increasing the number of classifiers in a random forest.

FIG. 14A is a graph of an example embodiment of the performance of all the classifiers for predicting a primary diagnosis.

FIG. 14B is a graph of an example embodiment of the performance of all the classifiers where agreement with either the primary or the alternative diagnoses is considered to be accurate.

FIG. 15 is a block diagram of an example internal structure of a computer optionally within an embodiment disclosed herein.

### DETAILED DESCRIPTION

A description of example embodiments follows.

While example embodiments disclosed herein are directed to the field of orthodontics, it should be understood that the same or similar embodiments can be directed to other areas of the medical field that involve multiple options for treatment diagnoses or treatment planning. It should also be understood that the example embodiments can be directed to fields outside of the medical field, such as machinery maintenance, including transportation vehicles and oil drilling rigs.

An example embodiment disclosed herein is an artificial intelligence (AI) based decision making model/system/platform for the diagnosis and treatment planning of orthodontic patients requiring extraction/non-extraction treatment. Unlike natural intelligence that is displayed by humans and animals, AI is intelligence demonstrated by machines. Machine learning is a form of AI that enables a system to learn from data, such as sensor data, data from databases, or other data. A focus of machine learning is to automatically learn to recognize complex patterns and make intelligent decisions based on data. Machine learning seeks to build intelligent systems or machines that can learn, automatically, and train themselves based on data, without being explicitly programmed or requiring human intervention. Neural networks, modeled loosely on the human brain, are a means for performing machine learning.

Embodiments may utilize machine learning methods selected from a group including: neural networks, logistic regression, random forest ensemble classifier, and customized decision-making expert systems (ES) to analyze data obtained from patient records, such as x-rays, photographs, and dental models, to provide a primary and secondary (i.e., alternative) treatment options out of, for example, 14 different treatment options. Embodiments are expandable to support more treatment options as they become available as science and technology advance the state-of-the-art treatments.

An example embodiment may combine an expert-decision making tree designed to limit possible choice sets in response to a larger variety of orthodontic variables, substantially increasing the accuracy of a computer-implemented method to predict a given treatment plan in a resolved manner. An example embodiment may process data obtained from patient x-rays, images, and/or models to identify features for accurate prediction of an optimal orthodontic diagnosis and treatment plan.

An example embodiment disclosed herein may be utilized by orthodontists, dentists, residents, and dental students for: a) diagnosis and treatment planning; b) an educational/e-learning tool; or c) a confirmatory tool for second-diagnosis to avoid potential irreversibility of an incorrect orthodontic treatment plan.

Currently, evaluating patient records, including x-rays, photographs, and teeth models, is subjective. It is often based on current knowledge, experience, beliefs, educational background, tradition, etc. Even though standardization and ground rules exist in identification of key features in patient records, the interpretation of the feature variables is entirely subjective. This can result in unknown, but widely acknowledged, errors in treatment planning with the potential of causing irreversible changes in a patient's jaw structure and facial physiognomy.

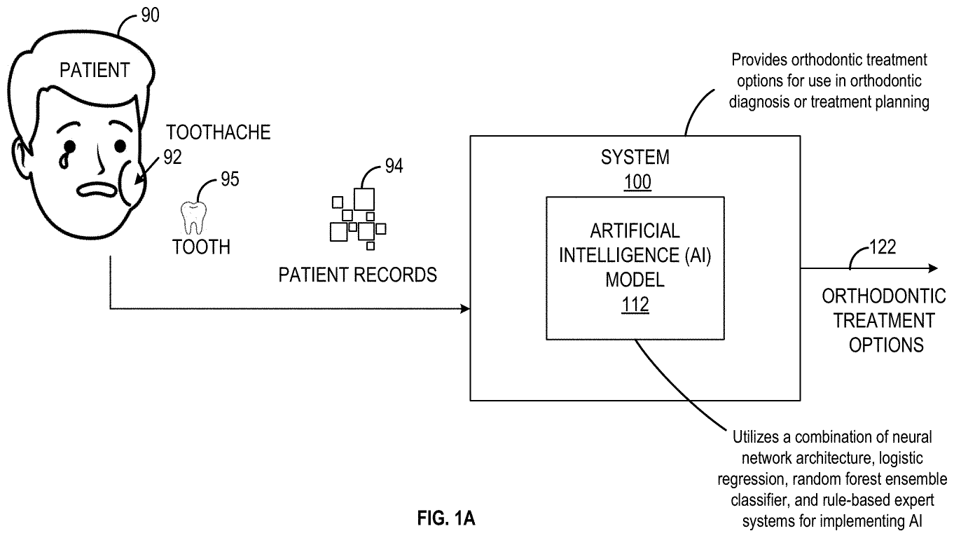

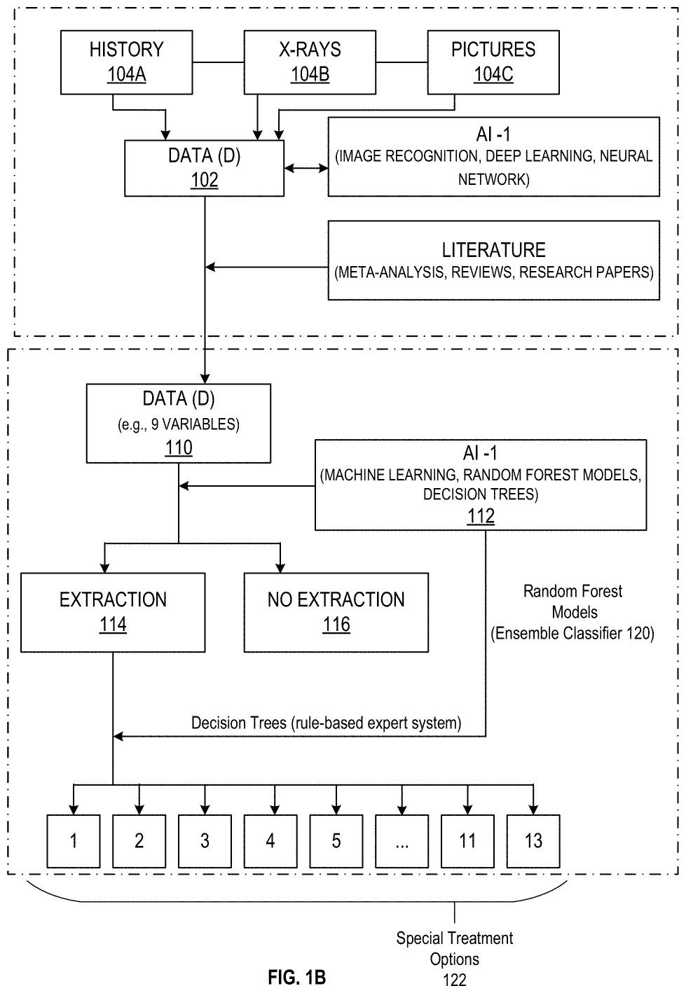

Orthodontic diagnosis is a time-consuming job of landmark identification, analysis and interpretation of patient photographs, dental study models, and x-rays. The final diagnosis interpreted from the gathered data is primarily based on a clinician's heuristics. Although these heuristics are based on pedagogical case presentation and gained experience, there is a lack of objective decision-making methodologies to arrive at a given (or a given set) of treatment plans in a consistent and accurate manner. A wrong decision can lead to undesirable results, such as suboptimal esthetics, improper bite, functional abnormalities related to mastication and speech and, in the worst-case scenario, an unfinished treatment. An example embodiment disclosed herein may process data obtained from patient x-rays, images, and/or models to identify features for accurate prediction of an optimal orthodontic diagnosis and treatment plan for a patient, such as the patient 90 of FIG. 1A, disclosed below.

FIG. 1A is a block diagram of an example embodiment of a system 100 that provides orthodontic treatment options for use in orthodontic diagnosis or treatment planning. In the example embodiment, a patient 90 is experiencing a toothache 92. According to an example embodiment, the system 100 processes data obtained from patient record(s) 94 of the patient 90. The patient record(s) 94 may include patient photographs, dental models, and/or x-rays of a tooth 95 or teeth (not shown) of the patient 90. The system may employ an artificial-intelligence (AI)-based decision-making model 112 that simulates an expert's decision of whether teeth need to be 1) extracted or 2) not extracted and outputs an orthodontic treatment option(s), as disclosed further below with regard to FIG. 1B. According to an example embodiment, the AI-based decision-making model 112 may utilize a neural network architecture, logistic regression, random forest ensemble classifier, and rule based expert systems in combination to implement the AI, as disclosed further below.

Example embodiments disclosed herein may employ an expert decision tree based on known expertise, literature, and expert opinions to create a consensus method to arrive at a set of decisions. This decision tree is then integrated with an AI-based method that further resolves a possible treatment plan based on collected expert data. This has resulted in accurate prediction of one among 14 different treatment plans. More or fewer treatment plans can also be used for the prediction.

Some major benefits of example embodiments disclosed herein include:

1. Elimination of variability in the decision-making process both inter-operator and intra-operator.
2. Considerable time savings as data processing and interpretation is completely automated.
3. Considerable reduction in misdiagnosis, which is the biggest source of malpractice lawsuits.
4. Potential as a powerful educational tool.
5. Independent improvement of prediction accuracy by adding new patient records as templates, just as a clinician might increase his/her clinical knowledge and experience. This means the model becomes more robust with time.
6. Until systems that employ embodiments disclosed herein have been adopted as the primary tool for diagnosis, an example embodiment can serve as a confirmatory/second-opinion tool to increase reliability of the primary treatment plan, reduce reliance on clinician subjective heuristics, and combine a wider expert opinion base into the treatment plan.

### EXAMPLE FEATURES

1. An example embodiment predicts an extraction or non-extraction treatment option by utilizing only nine parameters/variables with an accuracy of >90% (see attached research data). This is a dramatic reduction in the number of variables required to arrive at a decision. Current methods utilize many more variables to arrive at the same decision requiring more data. This can decrease accuracy.

2. Going forward, embodiments disclosed herein may require even fewer than the nine parameters to arrive at the binary decision of extraction/non-extraction treatment option. This will help in further reducing time and complexity of data recording, analysis, and interpretation.

3. Within extraction, an example embodiment also predicts a specific extraction treatment option out of the thirteen different treatment options by integrating ‘two’ expert system (ES) and random forest ensemble classifier. The accuracy is more than 70%. This has never been attempted before in any type of decision-making model. An example embodiment disclosed herein not only uniquely achieves this, but is able to predict a treatment option accurately in a highly resolved manner, for example (thirteen different treatment plans, instead of what is typically attempted in the binary: remove or not remove a tooth) from a minimal set of input variables.

4. An embodiment disclosed herein is an AI-based decision-making model that utilizes a combination of neural network architecture+Logistic regression+Random forest ensemble classifier+Rule based expert system (2)+Rule based expert system (10) seamlessly integrated into one broad application.

5. Finally, the integration of ES and Random forest ensemble classifier allows accurate and resolved (e.g., 1 among 14) prediction of treatment options from a small set of variables that can, in the near future, be obtained from AI-based image recognition. Dependence on a small set of variables provide a unique position for detect these parameters automatically from x-ray and patient images alone, creating a potential for an image-based prediction of an orthodontic treatment option.

The decision to extract teeth is one of the most critical and controversial in orthodontic treatment, largely because extractions are irreversible. These decisions are based on clinical evaluations, photographs, dental models, x-rays and on the experience and knowledge of the clinician. A wrong decision can lead to undesirable results, such as suboptimal esthetics, improper bite, functional issues in mastication and speech, and, in the worst-case scenario, unfinished treatment. Since there is no set formula, the decision depends on the practitioner's heuristics. This often causes intra-clinician and inter-clinician variability in the decision-making process. Additionally, for thousands of students, residents, orthodontists, and dentists across the globe, diagnosis and treatment planning can be very challenging. The gap in knowledge or data interpretation can be critical. An example embodiment disclosed herein provides an AI-based technology/model that addresses this problem from interpretation of patient data to arrive at a rationale diagnosis.

Machine learning methods have witnessed tremendous growth in data processing and analysis by making use of convolutional neural network systems. This emulates human learning in a situation that cannot be formulized or standardized. To date, however, there is no mathematical model that automatically interprets the patient records, analyzes the data, and simulates the orthodontic tooth-extraction/non-extraction decisions that would logically lead to a guaranteed optimum treatment outcome. An example embodiment disclosed herein is a decision-making model that simulates an expert's decision of whether teeth need to be 1) extracted or 2) not extracted based on standardized orthodontic pretreatment records (patient photographs, dental models, and x-rays). Such a decision-making model is included in the workflow 101 of FIG. 1B, disclosed below.

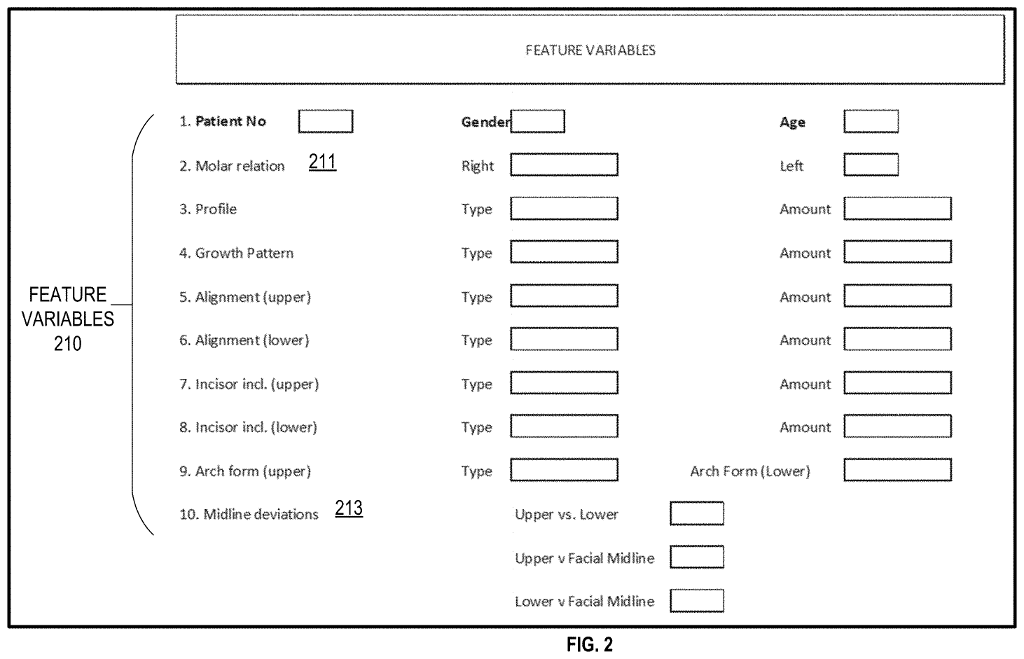

FIG. 1B is an outline of a workflow that simulates an expert's decision of whether teeth need to be 1) extracted or 2) not extracted. In the example embodiment, the data (D) 102 is obtained from a patient's history 104a, x-rays 104b, and pictures 104c and is categorized into 9 distinct clinical variables 110, such as shown in FIG. 2, disclosed below. It should be understood, however, that a number of the clinical variables 110 is not limited to 9.

FIG. 2 is a patient record that includes a set of feature variables 210 that may be employed as the clinical variables 110 of FIG. 1B, disclosed above.

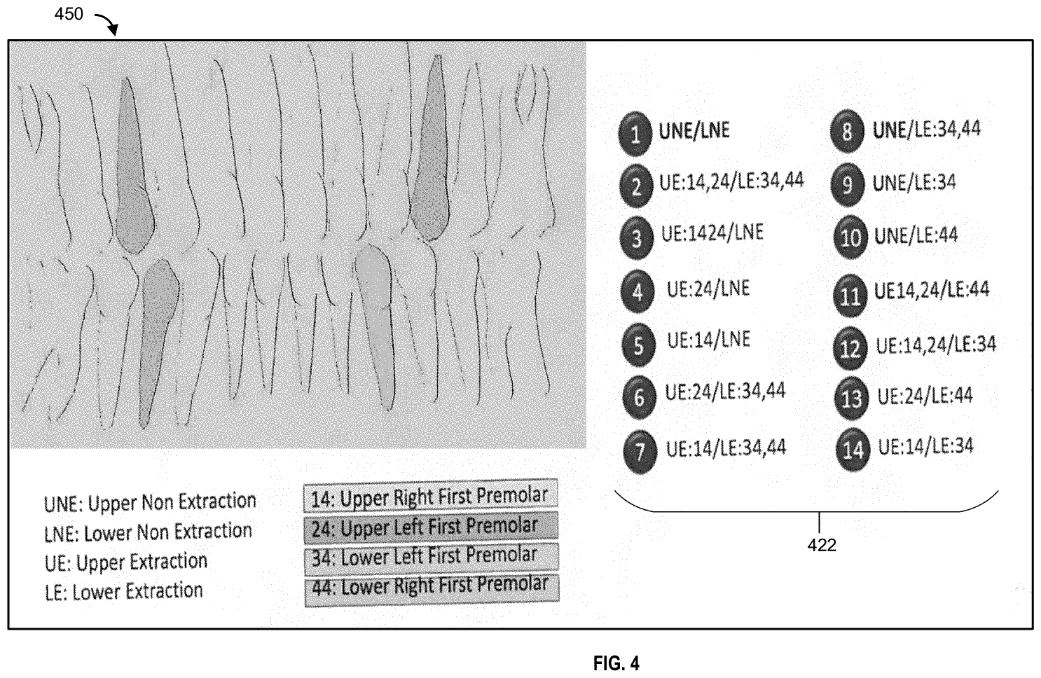

Referring back to FIG. 1B, the feature variables 110 are analyzed by an example embodiment of a model 112 disclosed herein to generate the correct treatment option, i.e., extraction 114 or non-extraction 116. The accuracy of the model 112 is >90%, based on research data disclosed further below, when compared to a panel of 6 expert orthodontists with considerable experience in the specialty of orthodontics. The model 112 works on neural network-based machine learning for data analysis and diagnosis and utilizes logistic regression 118 and a random forest model 120, such as an ensemble classifier. Within the extraction treatment option 114, there are 13 specific treatment options 122 depending on which combination of teeth need to be extracted, such as disclosed further below with regard to FIG. 4.

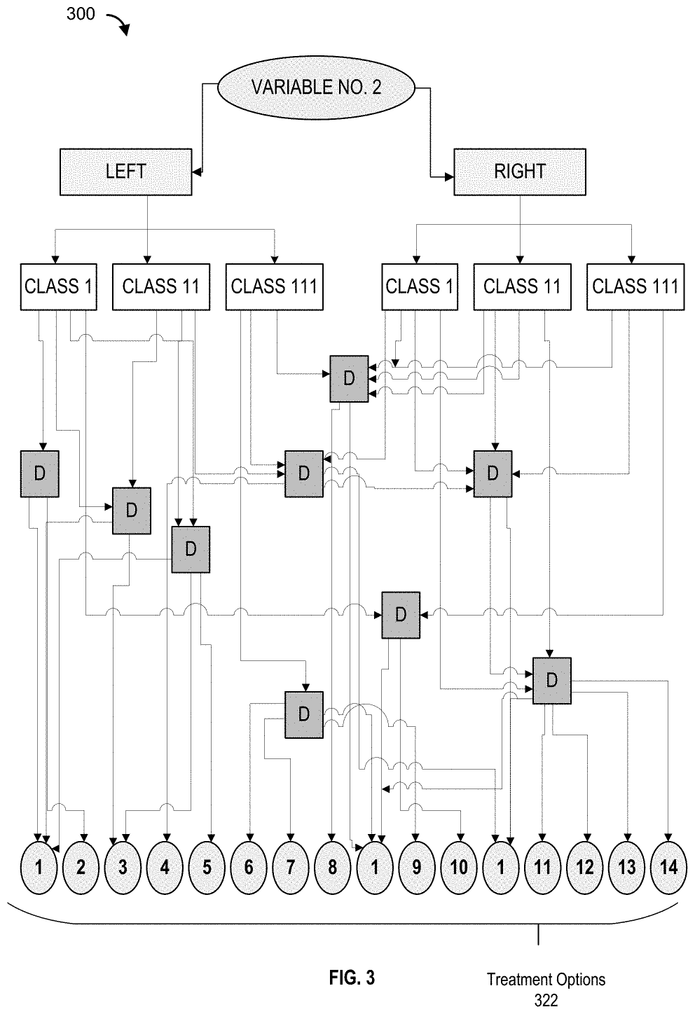

It should be understood that the number of specific treatment options 122 is not limited to 13, as illustrated in FIG. 1B. An example embodiment may be modified to support more or fewer treatment options as the treatment options change. For example, based on variable no. 2 (molar relation) 211 of FIG. 2, disclosed above, the model 112 of FIG. 1B may further interpret the specific set of tooth or teeth that need to be extracted, such as is shown in FIG. 3, disclosed below.

FIG. 3 is a decision diagram 300 of an example embodiment of decisions of a model for determining a treatment option based on a given feature variable, that is, variable no. 2 (molar relation) 211 of FIG. 2, disclosed above.

Referring to FIGS. 1B, 2, and 3, based on the variable no. 2 (molar relation) 211 feature variable, the model 112 further interprets the specific set of tooth or teeth that need to be extracted, such as shown in FIG. 3, by utilizing a rule based, decision-tree type expert system specifically developed to support the machine learning method. An expert system emulates the decision-making ability of a human expert and is designed to solve complex problems by reasoning through bodies of knowledge, represented mainly by rules rather than through conventional procedural code. In the example embodiment of FIG. 3, the model 112 automatically centers down upon 1 to 3 treatment options out of 14 treatment options 322 for a particular patient. When there is a situation where two extraction treatment options are presented, variable no. 10 (midline deviations) 213 of FIG. 2 is utilized to narrow upon a single treatment option utilizing a second rule based expert system.

The model for specific treatment plan revealed a >70% accuracy, based on research data as disclosed further below. This moves up to >90% when the model is given the option of picking the top two choices of treatment. This level of specificity has not been attempted in any of the previous machine or non-machine learning based models.

An example embodiment disclosed herein can synch together a number of AI-based applications: Neural network architecture+Logistic regression+Random forest ensemble classifier+Rule based expert system (variable 2)+Rule based expert system (variable 10), to create a decision-making model for orthodontic diagnosis and treatment planning. Such an approach makes the final model robust and helps it go beyond the binary treatment option of extraction or non-extraction. This integration allows for a highly resolved and accurate prediction of a treatment plan from a very small set of input variables, which could be potentially be derived automatically from patient x-rays and images only, such as the image of the teeth 450 of FIG. 4, disclosed below.

FIG. 4 is an image of an example embodiment of teeth 450. The image of the teeth 450 shows the locations of the upper and lower premolars. The 14-options to the right of the image of the teeth 450 lists the specific extraction procedures 422 with corresponding indices and in terms of the locations of the teeth, where “NE” refers to no extraction.

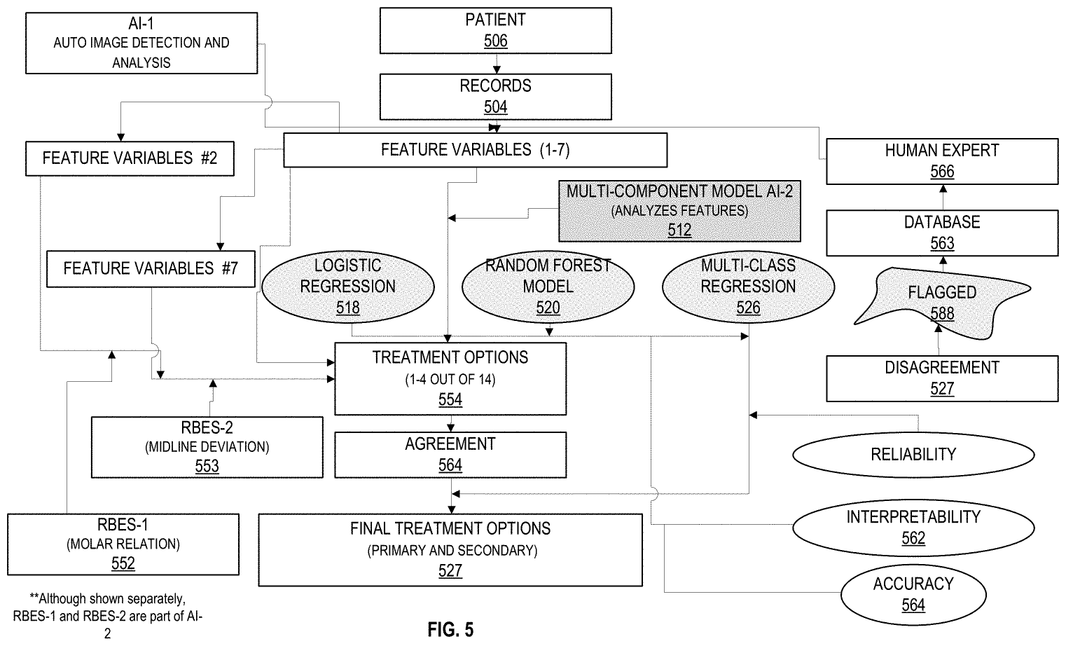

Referring back to FIG. 1B, according to an example embodiment, the model 112 may be an AI-based decision-making model that utilizes a multi-component model that may include a two layer neural network architecture, logistic regression model, and random forest ensemble classifier in combination with Rule based expert systems (RBESs), seamlessly and systematically integrated into one broad application, such as disclosed below with regard to FIG. 5.

FIG. 5 is a block diagram of example embodiments of methods for providing orthodontic treatment options for use in orthodontic diagnosis or treatment planning. Described below with regard to FIG. 5 are four example methods to solve the specific problem of orthodontic diagnosis and some related open problems in medical machine learning such as safety, accuracy and interpretability.

a. Multiple Rule Based Expert Systems (RBESs): An example embodiment employs a set of rules strategically placed in the computer implemented method, such that the data can be channeled first through the RBESs before arriving at the AI interface. An example embodiment utilizes two RBESs 552, 553, which have been specifically designed (custom made) from clinical experience and research to help the system select 554 one to four treatment (tx) outcomes out of the 13 possible. As this technology expands, more RBESs can be created for automatic diagnosis of other orthodontic problems. The RBESs 552, 553 help in 1) reducing computing power, 2) reducing errors, 3) increasing accuracy of prediction, and 4) increasing resolution of hierarchical prediction trees.

b. Multi-component AI model 512 for interpretable learning: Another example embodiment employs a unique combination of 1) a logistic regression model 518 to provide an interpretable model predicting extraction vs. non-extraction treatment option, 2) a multiclass logistic regression (two-layer neural network) 526 to provide an interpretable model to predict the ‘specific’ type of extraction treatment option, and 3) a random forest model 520 to provide both an accurate and robust prediction, for solving the specific problem of orthodontic diagnosis.

The first two models are easily interpretable, though slightly less accurate than the random forest model 520, but can provide guidance to users when they are not sure why the system is suggesting a particular treatment. This way, the system uniquely provides both interpretability 562 and accuracy 564 of tooth-extraction prediction, which are usually two conflicting goals.

c. Qualitative description for numerical values to mimic the human brain: Another example embodiment provides a new qualitative description for every parameter mimicking ‘real world’ scenarios, i.e., the method by which a clinician/orthodontist 566 will interpret patient records 504. The traditional approach of utilizing numerical values has been discarded as it only carries academic importance.

As disclosed, all possible numerical outputs have been clubbed into 2-3 broad qualitative categories, such; high, average, low or severe, moderate, low. Each category is made to capture a certain range of numerical values. This approach relies on learning ‘qualitative patterns’ in the input data, rather than relying on numerical values that might not capture the true characteristics, and are generally unavailable in regular orthodontic practice.

d. An example embodiment in which the rule based expert systems (RBESs) 552, 553 are seamlessly integrated with the multi-component AI model 512: Combining the RBESs 552, 553 with the multi-component model 512 provides a ‘safety check,’ such that if they do not broadly agree upon the Tx options (i.e., there is a ‘disagreement’ 527), then the system rejects the Tx options 554, flags 558 them, sends them to a database 563 for analysis by a human expert(s) 566, such as orthodontists. If both are in ‘agreement’ 564, the multi-component model 512 further analyzes the Tx options 554 and suggests a primary and secondary Tx option 567. This feature has the following uses:

1) Extra layer of security/reliability by constraining the Tx options to a select few to which both RBESs 552, 553 and Multi-component model 512 have to ‘agree.’

2) Elimination of major errors: safety check. This is a ‘major’ advantage as wrong decisions can be very costly.

3) The ‘disagreements’ are directed back to a group of qualified human experts (orthodontists) 566 for resolution. The outcome is automatically added to a database 563 from which the system can learn, adapt, and become better at interpreting outliers.

4) The above helps create a ‘weighted’ AI decision model as the multi-component model 512 communicates with the RBES interface for future updates.

Value Proposition

An example embodiment may serve as a primary diagnostic tool for dentists. The example embodiment can add considerable value in rendering orthodontic care:

1. Elimination of variability in the decision-making process both inter-operator and intra-operator.

2. Saving considerable time as data processing and interpretation is completely automated.

3. Considerable reduction in misdiagnosis. It is the biggest source of malpractice lawsuits.

4. Potential as a powerful educational tool.

5. It can independently improve its prediction accuracy by adding new patient records as templates, just as a clinician might increase his/her clinical knowledge and experience. This means the model becomes more robust with time.

6. Aligner companies are spending millions of dollars to hire orthodontic consultants for identifying patients that can be treated with aligners. This function can be completely automated by an embodiment of the invention, eliminating the need for an orthodontic consultant.

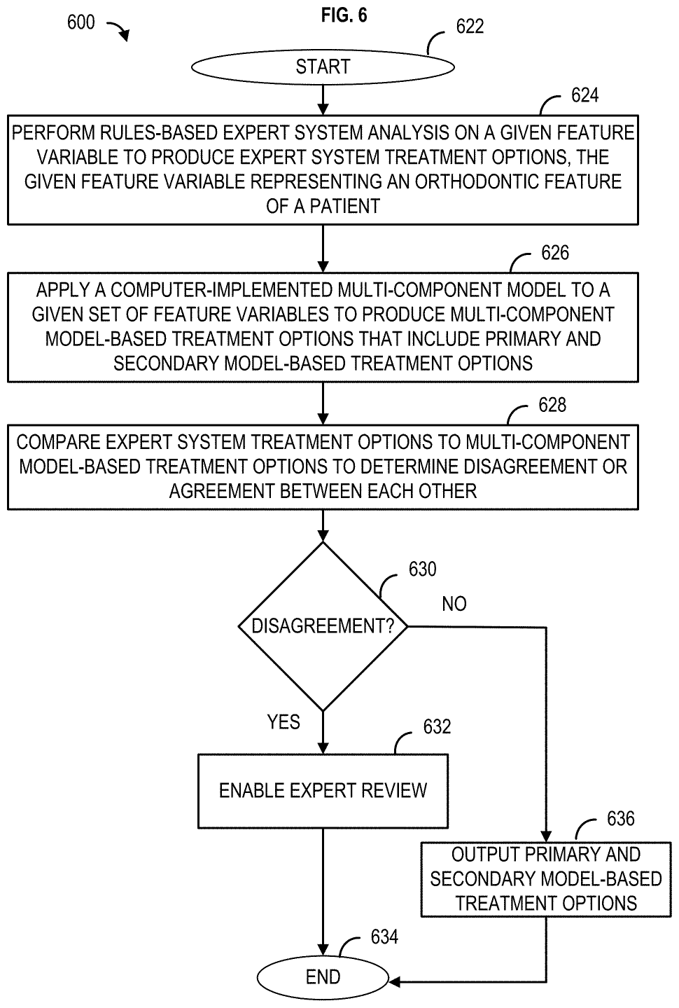

Example embodiments disclosed herein can make a significant impact on the safety, interpretability, and accuracy of orthodontic diagnosis of extraction/non-extraction tx approaches by using the proposed AI system. All of the above example embodiments are distinguished over existing technology in the specialty of orthodontics for the purpose of orthodontic diagnosis. Embodiments disclosed are based on an in-depth expertise of orthodontics at the research, clinical and academic levels, in combination with specific knowledge of relevant areas of statistics and machine learning. Embodiments disclosed herein may be implemented in the form of an apparatus, system, or computer readable medium with program codes embodied thereon, or method, such as the method of FIG. 6, disclosed below.

FIG. 6 is a flow diagram 600 of an example embodiment of a computer-implemented method for providing orthodontic treatment options for use in orthodontic diagnosis or treatment planning. The method begins (622) and performs a rules-based expert system analysis on a given feature variable to produce expert system treatment options, the given feature variable representing an orthodontic feature of a patient, the expert system treatment options being fewer in number than a number of standard orthodontic treatment options that apply to orthodontic diagnosis, orthodontic treatment options, or both (624). The method applies a computer-implemented multi-component model to a given set of feature variables to produce multi-component model-based treatment options that include primary and secondary model-based treatment options (626). The method compares the expert system treatment options to the multi-component model-based treatment options to determine disagreement or agreement between each other (628). If a check for disagreement (630) is yes, the method further enables an expert to review the expert system treatment options and the multi-component model-based treatment options, adapts at least one of the given feature variable, rules-based expert system analysis, or multi-component model based on feedback from the expert (632), and the method thereafter ends (634) in the example embodiment. If, however, the check for disagreement (630) is no, then there is agreement and the method outputs the primary and secondary model-based treatment options to a clinician (636) and the method thereafter ends (634) in the example embodiment.

A system implementing the method is used to train dentists or orthodontists in diagnosis, treatment planning, or a combination thereof. An analysis or final result provided by the method may be reviewed and approved by a certified orthodontist or other expert medically and legally qualified in the relevant jurisdiction(s) to recommend treatment/diagnosis.

As disclosed above, extraction of teeth is an important treatment decision in orthodontic practice. An expert system that is able to arrive at suitable treatment decisions can be valuable to clinicians for verifying treatment plans, minimizing human error, training orthodontists, and improving reliability. As disclosed below, a number of machine learning models were trained for this prediction task using data for 287 patients, evaluated independently by 5 different orthodontists. The following discloses why ensemble methods are particularly suited for this task. The performance of the machine learning models is evaluated and training behavior interpreted. Results for an example embodiment of a model disclosed herein are close to the level of agreement between different orthodontists.

As disclosed above, extraction of teeth is one of the most critical and controversial decisions in orthodontic treatment, largely because extractions are irreversible. (Weintraub J A, Vig P S, Brown C, Kowalski C J. The prevalence of orthodontic extractions. Am J OrthodDentofacial Orthop 1989; 96: 462-6; Burrow, S. J.: To extract or not to extract: A diagnostic decision, not a marketing decision, Am. J. Orthod 2008; 133:341-42). These decisions are based on clinical evaluations, patient photographs, dental study models, radiographs, and substantially rely upon the experience and knowledge of the clinician. A wrong decision can lead to undesirable results like suboptimal esthetics, improper bite, functional abnormalities related to mastication & speech and in the worst-case scenario, an unfinished treatment. Till date, decision to extract teeth is not formalized and standardized, and depends upon the practitioner's heuristics. (Ribarevski R, Vig P, Vig K D, Weyant R, O'Brien K. Consistency of orthodontic extraction decisions. Eur J Orthod 1996; 18:77-80.) This often causes intra-clinician and inter-clinician variability in the decision-making process (Dunbar A C, Beam D, McIntyre G. The influence of using digital diagnostic information on orthodontic treatment planning-a pilot study. J Healthc Eng 2014; 5:411-27; Baumrind S, et al. The decision to extract: Part 1—Inter-clinician agreement. Am J Orthod Dentofac Orthop1996; 109:297-309). Therefore, for hundreds of students, residents, orthodontists and dentists across the globe diagnosis and treatment planning poses a significant challenge. The resultant gap in the knowledge or data interpretation can be critical. Therefore, in order to standardize the decision-making process newer approaches are required.

An example embodiment creates an artificial intelligence decision-making model for the diagnosis of extractions using neural network machine learning. The primary objectives of a study disclosed herein were (1) to develop a decision-making model that simulates experts' decision of whether a teeth need to be extracted or not based on standardized orthodontic pretreatment records (patient photographs & x-rays), and (2) to determine the knowledge elements required in formulating orthodontic extraction/non-extraction treatment decisions. It was expected that the diagnostic model created would match an expert's diagnosis, both in binary decision making (extraction vs non-extraction outcomes), and in the more resolved decision-making process of which specific extraction outcome would be followed (out of the 13 possible outcomes). This method would not only limit variability in decision making in orthodontics, but also limit the adverse effects of wrongly prescribed tooth extraction protocol. Additionally, this could also serve as a testing tool to train dentists & orthodontic students.

Orthodontic pretreatment records in the form of extraoral photos, intra-oral photos & cephalometric x-rays were collected. A panel of experienced orthodontists (also henceforth referred to as experts) evaluated the records individually and predicted the final outcome of extraction/non-extraction.

Materials and Methods

Data Collection and Feature Selection

The data consisted of 300 pretreatment patient records obtained from a private practice in Norwalk, Ohio, USA (orthodontist: C.A). Medical charts and conventional diagnostic records such as lateral head films (cephalometric x-rays), panoramic radiographs, facial photographs, and intraoral photographs, were employed for each subject and screened by C.A for completeness. All subjects had full permanent dentitions except for the third molar, no abnormalities of the craniofacial forms or skeletal deformities and no history of orthodontic treatment. Nineteen feature variables or elements that characterize orthodontic problems and assumed to be important in deciding whether or not teeth need to be extracted were selected. This selection was based on existing orthodontic literature. For all subjects, 5 experts (C.A, V.M, D.S, C.P.J,), with an average experience of approximately 9 years among them examined the records of each patient based on the pre-selected feature variables. Each expert also recorded his/her two most likely diagnostic outcomes (out of 14 available options) and categorized them as primary treatment & alternate treatment.

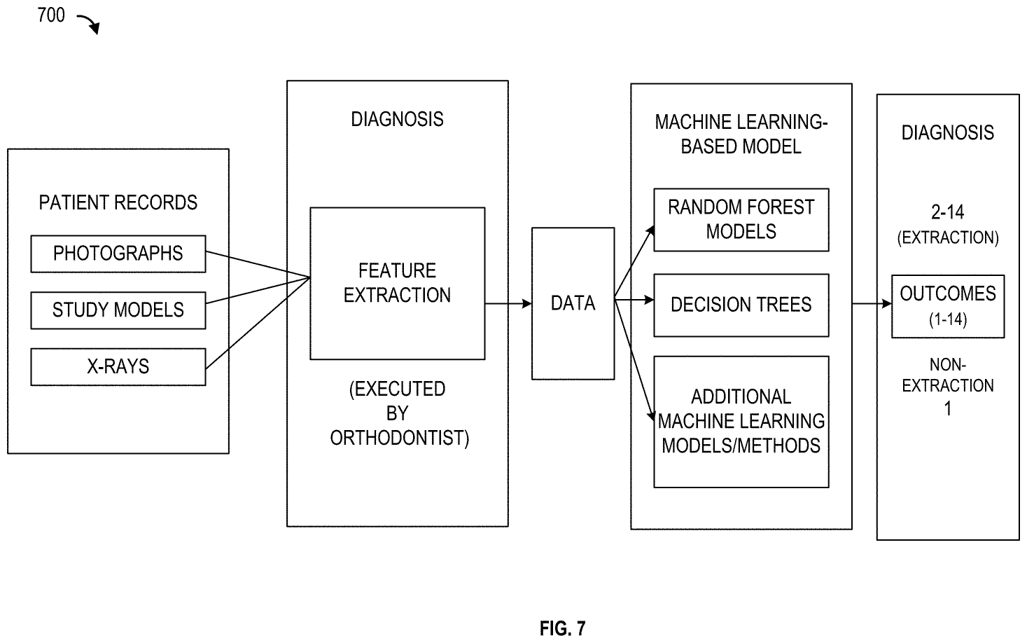

FIG. 7 is a block diagram of an example embodiment of a workflow from data collection to the machine learning diagnosis which was implemented. The data was compiled and evaluated for potential errors by one of the authors (U.M). Data sets for thirteen patients were eliminated due to incomplete records, & errors in data recording.

Computational Analysis

Expert provided features and decision data was analyzed using the R6 platform. The neural network model was built using the nnet package, while the random forests were built and evaluated using the RandomForest package. All calculations were performed using 5-fold cross validation. The same cross-validation set were used for each model and hyperparameter determination.

Results

Data for 287 patients from 5 different experts was collected. Each expert assigned values to 19 pre-selected diagnostic features based on cephalometric images and patient photographs in addition to selecting a primary and alternate treatment option. Experts were allowed to decide between one of the two binary outcomes: non-extraction, or extraction. Within the extraction plan depending upon which tooth/teeth required extraction, the experts had to select one (specific) outcome out of the 13 different options (2-14) provided in FIG. 4, disclosed above. Crucially, the experts also opined on the second most preferred outcome (termed alternative outcome), which considering the variability between expert's opinion, allowed for testing of the accuracy of an outcome based on example embodiment to be tested in a more robust manner.

Exploratory Analysis

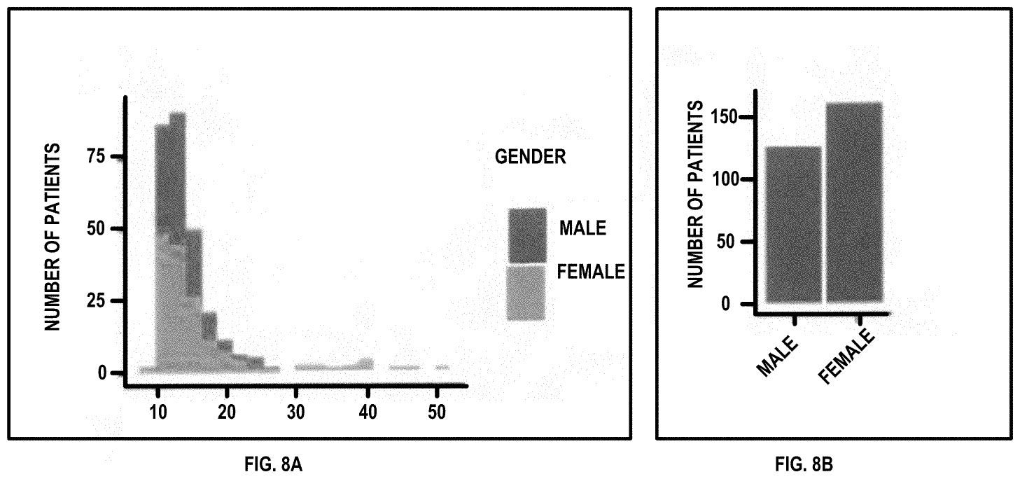

Patient data from 287 patients was used and the demographic data of the patients is disclosed in FIGS. 8A and 8B, below.

FIG. 8A is a graph of an example embodiment of the age distribution of the patients from whom patient data was taken for the study.

FIG. 8B is a graph of an example embodiment of the gender distribution of the patients from whom patient data was taken for the study.

First, the degree of agreement between the experts who evaluated the patients included in the study was established. If the multiple treatment plans selected by different experts are considered as the gold standard for a machine learning method, the inter-expert agreement should provide us a practical higher limit on the accuracy to achieve. The agreement on the primary outcome of treatment between different experts varied from 65% to 71% (Table 1), and agreement on either the primary or alternative outcome varied from 93% to 98% (Table 2). These data highlight that different experts, well trained in orthodontics, could defer in their primary opinions in some aspects. Tables 1 and 2 are disclosed below.

TABLE 1
Percentage agreement on the primary outcome of
treatment between different experts.

| | Expert 1 | Expert 2 | Expert 3 | Expert 4 | Expert 5 |
|---|---|---|---|---|---|
| Expert 1 | 100.0% | 71.1% | 64.8% | 68.3% | 69.0% |
| Expert 2 | 71.1% | 100.0% | 70.7% | 71.8% | 78.0% |
| Expert 3 | 64.8% | 70.7% | 100.0% | 63.8% | 69.7% |
| Expert 4 | 68.3% | 71.8% | 63.8% | 100.0% | 70.4% |
| Expert 5 | 69.0% | 78.0% | 69.7% | 70.4% | 100.0% |

TABLE 2
Percentage agreement on either the primary or alternative
outcome of treatment between different experts

| | Expert 1 | Expert 2 | Expert 3 | Expert 4 | Expert 5 |
|---|---|---|---|---|---|
| Expert 1 | 100.0% | 95.5% | 94.4% | 95.5% | 96.5% |
| Expert 2 | 95.5% | 100.0% | 95.5% | 95.1% | 96.5% |
| Expert 3 | 94.4% | 95.5% | 100.0% | 93.0% | 96.2% |
| Expert 4 | 95.5% | 95.1% | 93.0% | 100.0% | 97.9% |
| Expert 5 | 96.5% | 96.5% | 96.2% | 97.9% | 100.0% |

Machine Learning Model

Single Classifiers

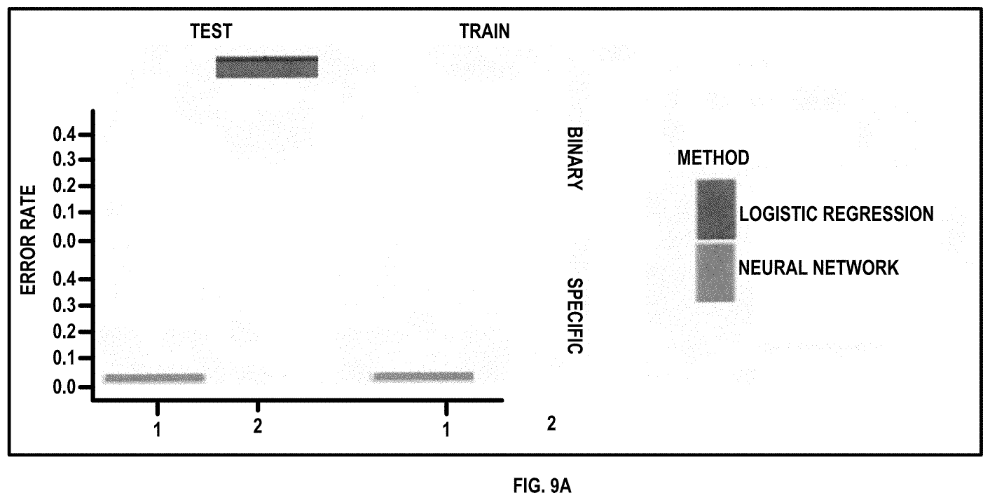

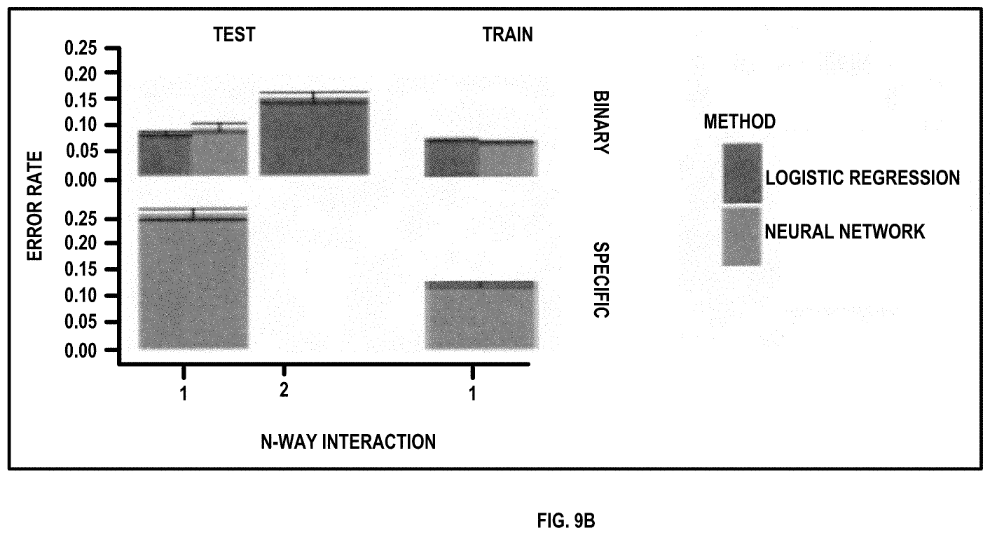

A number of different methods can be used to build a classifier for the prediction of orthodontic extractions. Twin problems of predicting whether to extract teeth or not, and the specific extraction treatment plan, were considered. As a classification problem, a discrete prediction was used and a neural network was used to learn the multinomial regression. Each output neuron learns to predict a specific extraction, taking inputs from the raw data. No hidden units were used. In addition, logistic regression was used for predicting the binary decision of extraction/non-extraction. FIGS. 9A and 9B, disclosed below, are graphs that show the performance of the logistic regression and the multinomial regression neural network model.

FIG. 9A is a graph that shows performance of a logistic regression model and a multinomial regression neural network model when considering only a primary diagnosis.

FIG. 9B is a graph that shows performance of a logistic regression model and a multinomial regression neural network model when considering both the primary and alternative diagnosis.

The logistic regression model, by definition, was not able to predict the specific extraction procedure. However, for the binary problem (extraction/non-extraction), the logistic regression outperformed the multinomial trained neural network.

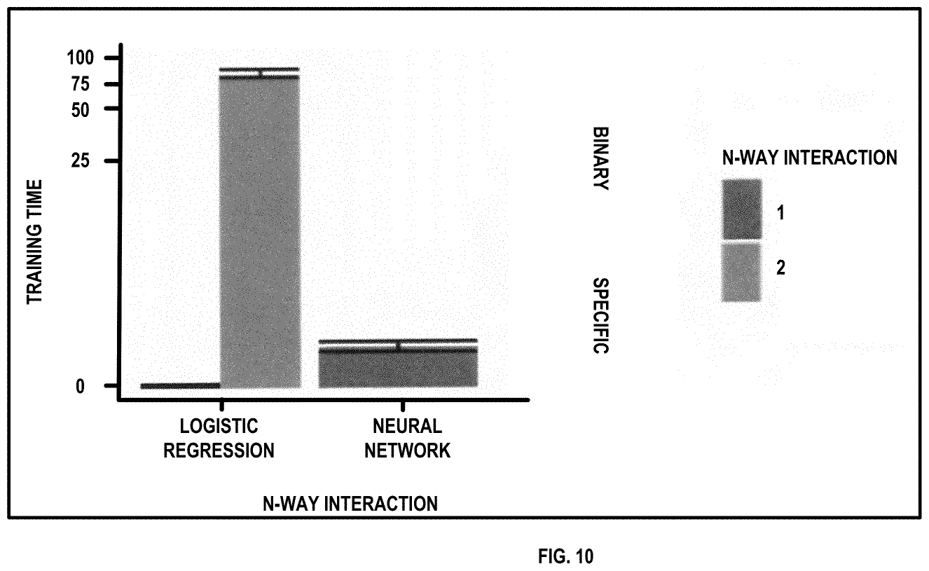

The next step towards increasing the performance of the model was the use of 2-way interactions in the logistic regression. Every pair of features were multiplied and used as additional features, generating a larger number of parameters. Although this was helpful in decreasing the error rates of the training sample, it increased the error of the test set, indicating that increasing the complexity of the model led to overfitting. The multinomial neural network model was not trained with 2-way interactions because firstly, the additional number of parameters in the neural network already predisposed it to overfitting, and secondly, the training time was most likely going to be inconvenient, as disclosed in FIG. 10.

FIG. 10 is a graph an example embodiment of training time for the single classifiers. Logistic regression was also trained with product terms (i.e., 2-way interactions), dramatically increasing the training time. Due to the large dynamic range needed on the y-axis, it is drawn in the pseudo-log transform.

Random Forest as an Ensemble Classifier

Since the addition of additional parameters in the classifier (as seen in the logistic regression with multiplicative terms) leads to overfitting, an ensemble of classifiers was used to improve the performance. Ensemble methods are known to be resistant to overfitting. Random forest models using the standard method were trained, and the main hyperparameters were varied to gain insight into the limitations for the performance.

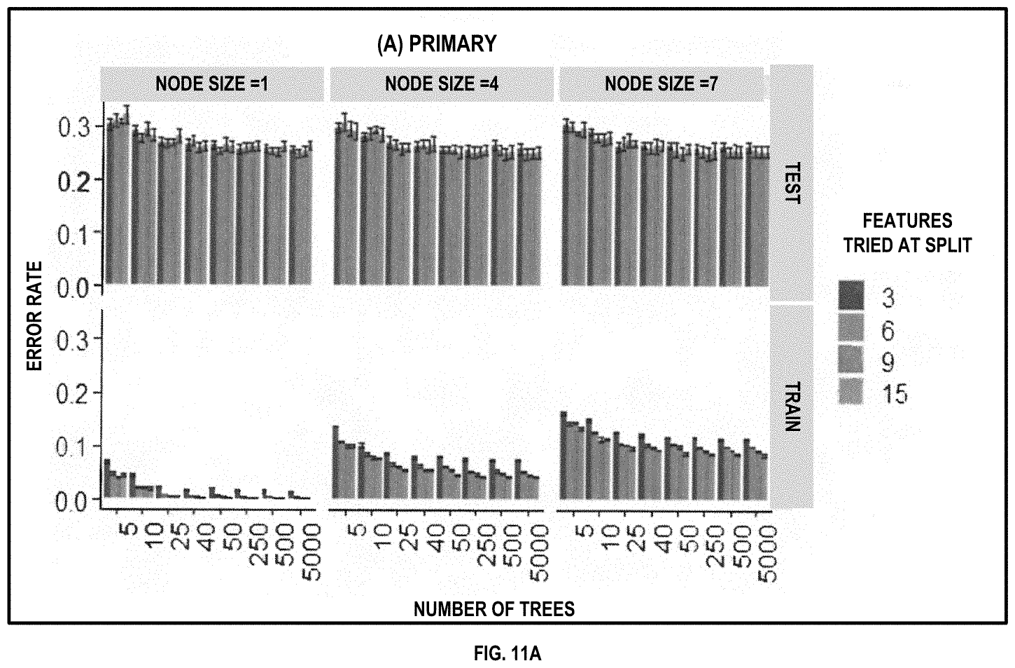

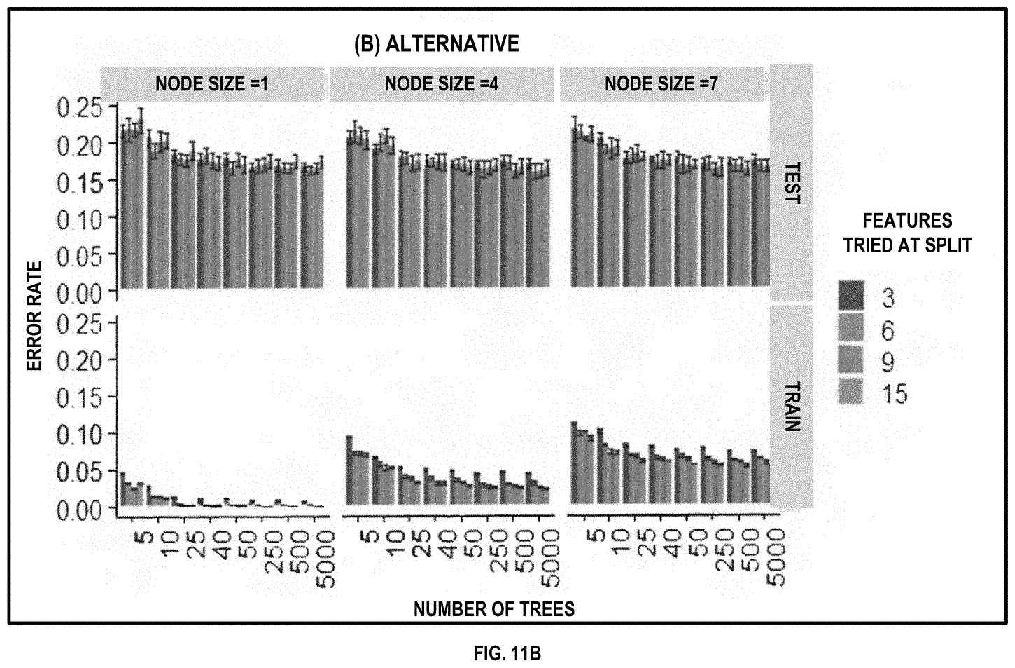

FIGS. 11A and 11B are graphs of an example embodiment of an effect of various training parameters on the Random Forest model for the prediction of the specific extraction. The minimum node size, features tried at every level of split, and the number of trees were varied and the error rates for the training and test split plotted.

Each decision tree in the random forest was constructed using a dataset sampled with replacement from the training set. This process of bagging is one of the ways in which each decision tree attempts to capture a different aspect of the data. During the construction of each decision tree, a small number of features were randomly selected at each level and the one that was the most discriminating among the classes was used. The process continued until each node contained no more than a specific minimal number of samples. These hyperparameters were varied during the training of the random forest model.

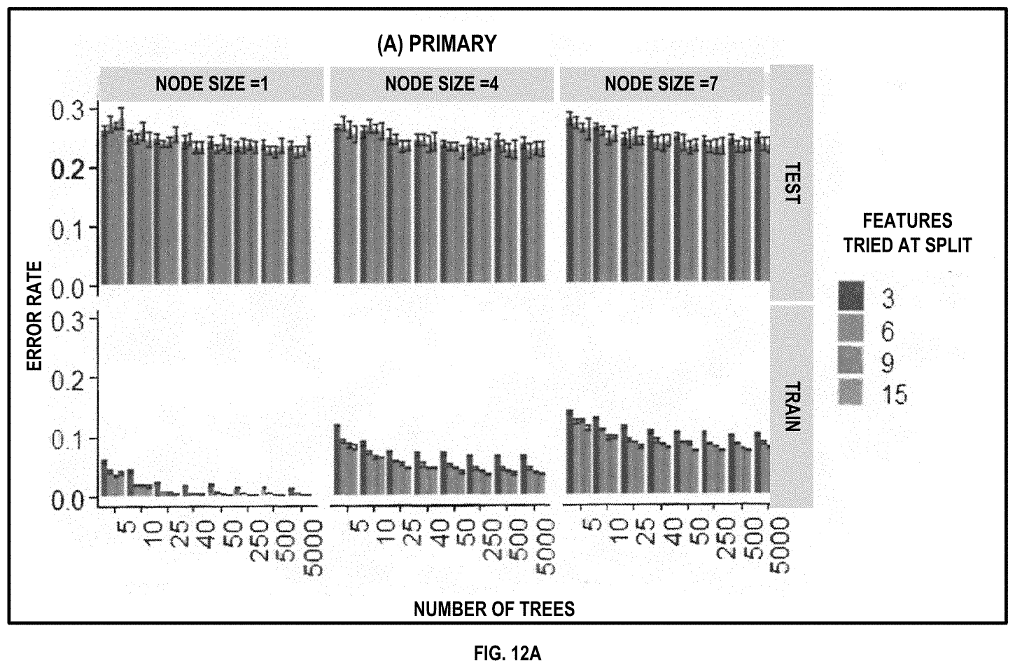

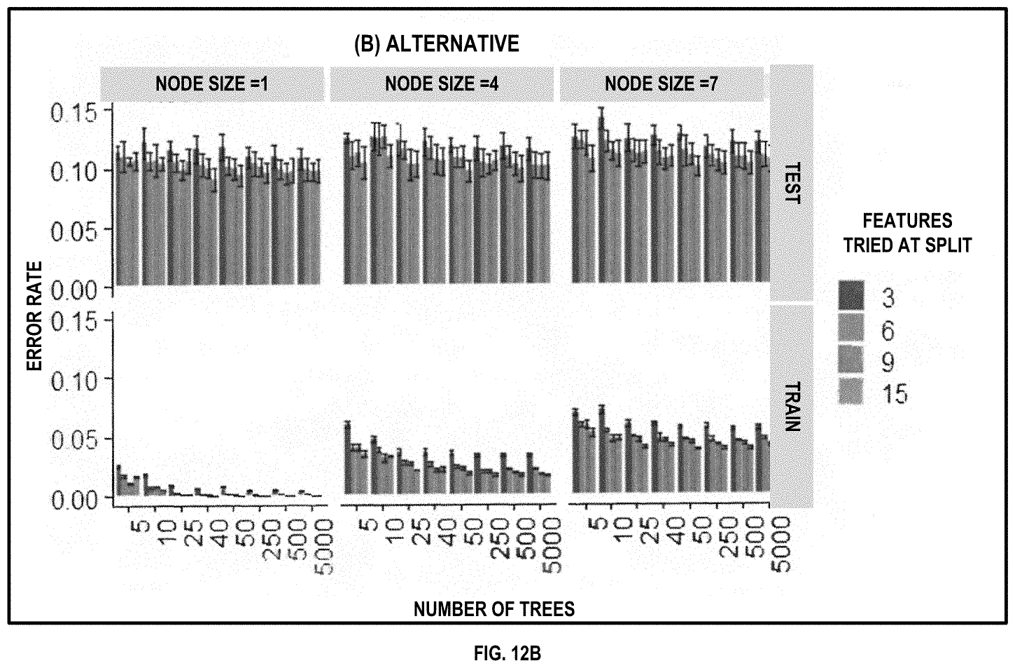

FIGS. 12A and 12B are graphs of an example embodiment of an effect of various training parameters on the Random Forest model for the binary predication problem. The minimum node size, features tried at every level of split, and the number of trees was varied and the error rates for the training and test split plotted.

FIGS. 11A and 11B and FIGS. 12A and 12B show the performance against a number of hyperparameters needed to fit the size of the available data. Observing the training data alone, it was evident that (a) performance was better for smaller minimal node sizes as it led to deeper decision trees, (b) the number of features at each split had an initial effect, but is saturated with increasing feature numbers, and (c) that even 50 trees showed a performance statistically indistinguishable from random forests with a much larger number of decision trees. Most notably, the prediction error showed no overfitting in test data (i.e., no increase in error rate was observed as the complexity of the model increased).

Also, even the relatively weaker hyperparameters (˜25 trees, a minimum node size of 4, and 6 features tried at every split) are strong enough to saturate the test set performance while the training set performance continues to decrease with more complex models. Similar behavior is seen when looking at the prediction of the specific extraction (FIGS. 11A and 11B) and the binary problem of predicting extraction vs. non-extraction (FIGS. 12A and 12B).

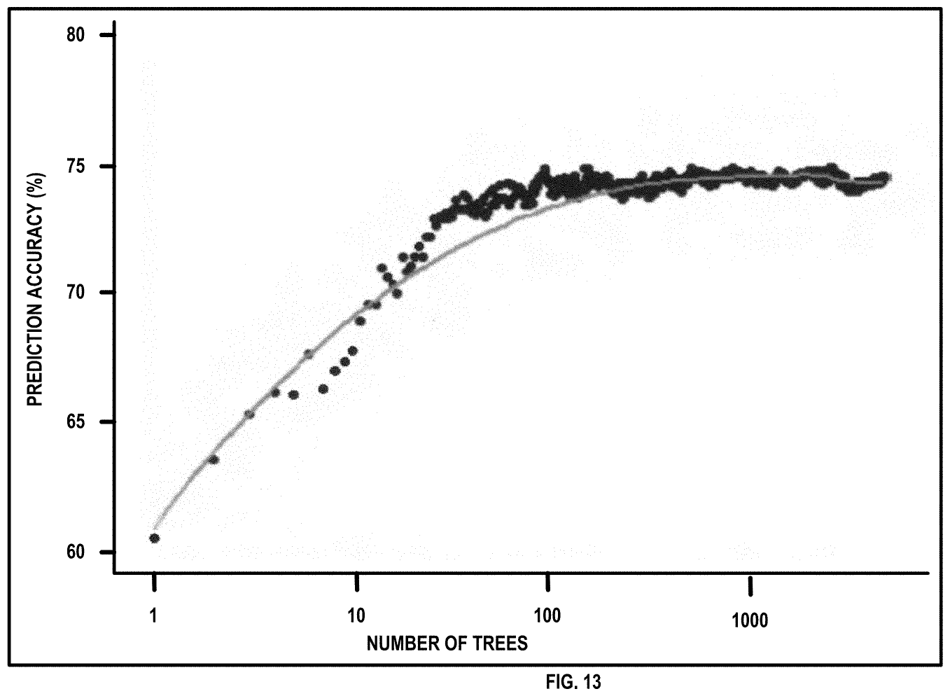

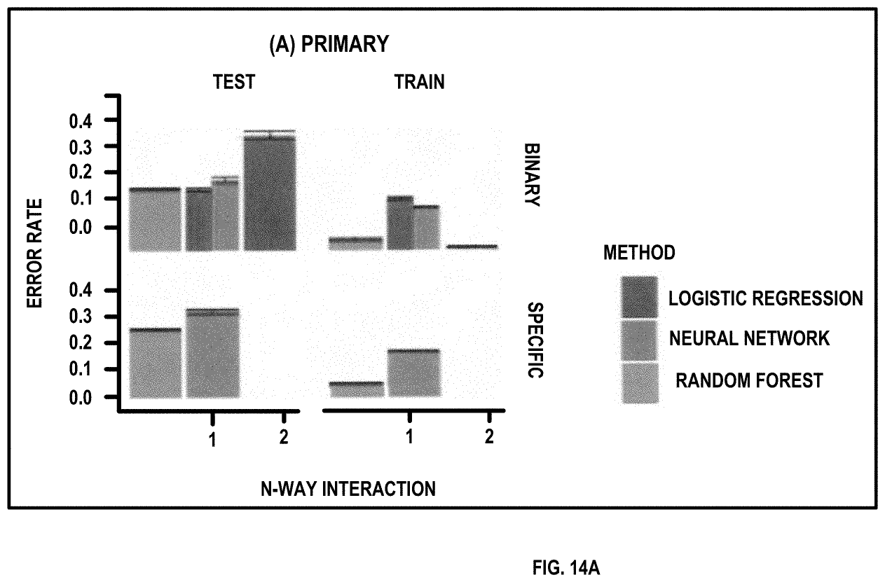

Since the random forest method has an out of bag data sample for the construction of each decision tree, this out of bag error rate can be used to study the effect of adding each additional tree. The out of bag accuracy, which is a proxy for the accuracy on the test set, is visualized in FIG. 13, that shows the saturation of the performance around 50 to 100 trees in the random forest model.

FIG. 13 is graph of a saturating effect of increasing the number of classifiers in a random forest. The out-of-bag accuracy (an estimate of the test accuracy) is plotted against the number of trees for the random forest model predicting the specific type of interaction. This is for a minimal node size of 1 and trying all possible features at every split.

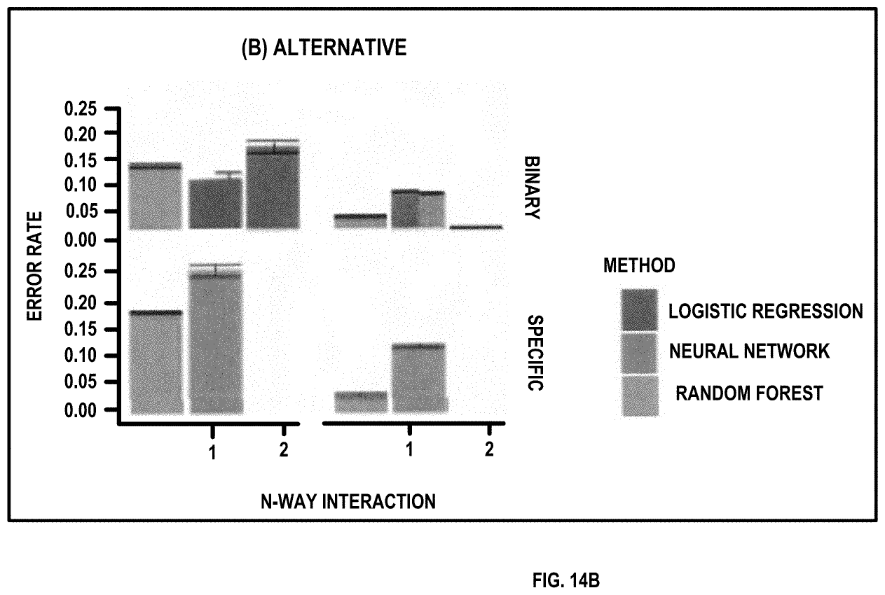

FIG. 14A is a graph of an example embodiment of the performance of all the classifiers for predicting the primary diagnosis.

FIG. 14B is a graph of an example embodiment of the performance of all the classifiers where agreement with either the primary or the alternative diagnoses is considered to be accurate. Here, both the single and ensemble (random forest) classifiers are included.

When comparing all classifiers (FIGS. 14A and 14B), it is clear that the Random Forest classifier outperforms the neural network model for the prediction of the specific extraction treatment. Logistic regression is able to achieve marginally better performance only for the case of binary prediction when considering both the primary and alternative diagnoses from the expert (top left panel of FIG. 14B).

Previous studies have approached this problem by utilizing machine learning using a neural network (Xie X, Wang L, Wang A. Artificial neural network modeling for deciding if extractions are necessary prior to orthodontic treatment. Angle Orthod 2010; 80:262-6; Jung S K, Kim T W. New approach for the diagnosis of extractions with neural network machine learning. Am J Orthod Dentofacial Orthop 2016; 149:127-33). However, these approaches have been limited due to various shortcomings. The models shown in the results have specifically focused on binary outcomes, i.e., extraction vs. non-extraction, without outlining which tooth or set of teeth need extraction. Expert data disclosed herein has showed, and is also generally believed, that this binary decision is a first order decision, and requires limited expertise when compared to the more resolved decision about which tooth, or a set of teeth, need to be extracted. Furthermore, the binary decision is determined by fewer parameters (crowding or tooth inclination), a much easier scenario, while a more resolved decision requires determination of parameters which are yet to be standardized, highlighting the challenges involved in deciding among many other possible outcomes.

Research disclosed herein not only focusses on this binary decision but also on the thirteen other possible outcomes which highlight the specific tooth/teeth requiring extraction, creating a new artificial intelligence-based method to predict a plan from among a large number of possible extraction plans (FIG. 4) based on the 19 feature elements.

Second, after conducting a thorough review of the existing literature, the diagnostic features were limited to 19 most relevant predictors. The feature vector-elements adopted can be broadly classified into five major categories, i.e., sagittal dentoskeletal, vertical dentoskeletal relationship, transversedental relationship, soft tissue relationship and intra-arch conditions. Similar studies (Xie X, Wang L, Wang A. Artificial neural network modeling for deciding if extractions are necessary prior to orthodontic treatment. Angle Orthod 2010; 80:262-6.; Konstantonis D, Anthopoulou C, Makou M. Extraxxtion decision and identification of treatment predictors in class I malocclusions. Prog in Orthod 2013, 14:47;1-8) have included many more features which have not only increased their computational requirements but also added redundancy in their data set. Moreover, a small number of features for each patient can be easily obtained from the standard records without utilizing special diagnostic approaches. Fewer features also means that the experts spend less time analyzing the records of each patient thereby making themselves available to analyze more samples, helping in the evaluation of the accuracy of an example embodiment of the method disclosed herein in relation to the inter-expert disagreement.

One of the limitations of this study was that the treatment outcomes were confined to non-surgical orthodontic procedures only. Also, atypical extraction patterns like; lower incisor extraction, second premolar extractions, extractions due to pathological reasons etc. were excluded. In the current optimized model, however, the elements that represented such features were not adopted. This is because the current study primarily focused on optimizing routine orthodontic diagnostic protocols.

Finally, though the current model may not yet suffice to achieve complete agreement with human judgments, it should be noted that it has an advantage in that the system can independently improve its prediction accuracy by adding new patient records as templates just as orthodontists might increase their clinical knowledge and experience. This means the model will become more robust clinically for making decisions for individual patient treatment.

The study disclosed above demonstrated that a limited feature set and machine learning method disclosed herein is able to predict the extraction procedure to an accuracy that is approximately equal to that obtained from different experts. The use of an ensemble classifier (random forest (Ho T K. Random Decision Forests. Proc. of the 3rd International Conference on Document Analysis and Recognition, Montreal, QC, 14-16 Aug. 1995. pp. 278-282. doi:10.1109/ICDAR.1995.598994)) model allowed overfitting to be obviated, as has been confirmed in many studies earlier (Dietterich T. G. Ensemble Methods in Machine Leaming. In: Multiple Classifier Systems. MCS 2000. Lecture Notes in Computer Science, vol 1857. Springer, Berlin, Heidelberg; Breiman L. Machine Leaming 2001; 45: 5. doi:10.1023/A:1010933404324;) Friedman J, Hastie T, Tibshirani R. Additive logistic regression: a statistical view of boosting (with discussion and a rejoinder by the authors). The annals of statistics. 2000; 28(2):337-407). Further, the study disclosed herein has shown that an ensemble of simpler models outperforms more complex models, such as a neural network for the problem disclosed herein. The use of bagged batch training and dropouts may help the neural network model to compete with the random forest model.

A random forest ensemble classifier that simulates orthodontic tooth extraction/non-extraction decision making was developed and confirmed to show a high performance, within the range of the inter-expert agreement.

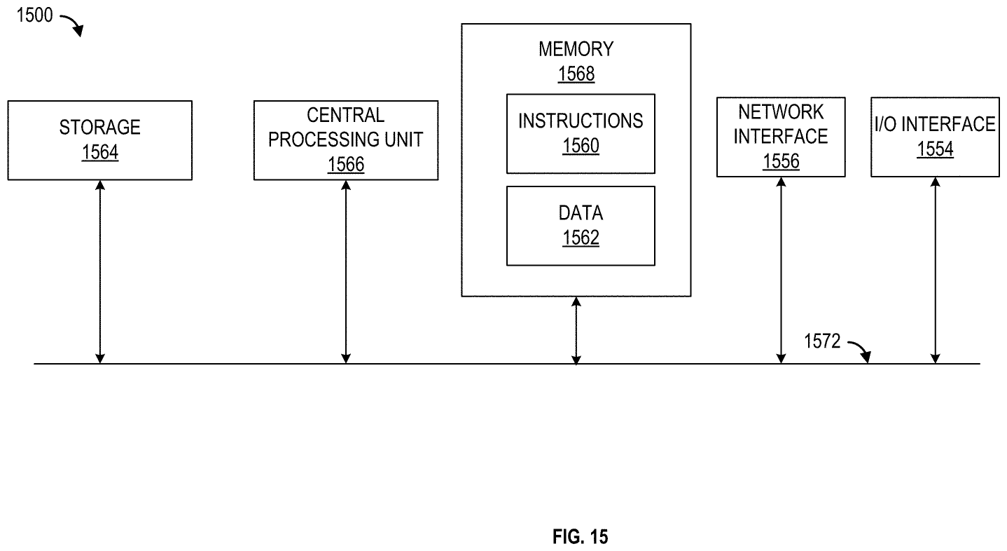

FIG. 15 is a block diagram of an example of the internal structure of a computer 1500 in which various embodiments of the present disclosure may be implemented. The computer 1500 contains a system bus 1552, where a bus is a set of hardware lines used for data transfer among the components of a computer or digital processing system. The system bus 1552 is essentially a shared conduit that connects different elements of a computer system (e.g., processor, disk storage, memory, input/output ports, network ports, etc.) that enables the transfer of information between the elements. Coupled to the system bus 1552 is an I/O device interface 1554 for connecting various input and output devices (e.g., keyboard, mouse, displays, printers, speakers, etc.) to the computer 1500. A network interface 1556 allows the computer 1500 to connect to various other devices attached to a network (e.g., global computer network, wide area network, local area network, etc.). Memory 1558 provides volatile or non-volatile storage for computer software instructions 1560 and data 1562 that may be used to implement embodiments of the present disclosure, where the volatile and non-volatile memories are examples of non-transitory media. Disk storage 1564 provides non-volatile storage for computer software instructions 1560 and data 1562 that may be used to implement embodiments of the present disclosure. A central processor unit 1566 is also coupled to the system bus 1552 and provides for the execution of computer instructions.

As used herein, the term ‘module’ may refer to any hardware, software, firmware, electronic control component, processing logic, and/or processor device, individually or in any combination, including without limitation: an application specific integrated circuit (ASIC), a field-programmable gate-array (FPGA), an electronic circuit, a processor and memory that executes one or more software or firmware programs, and/or other suitable components that provide the described functionality.

Example embodiments disclosed herein may be configured using a computer program product; for example, controls may be programmed in software for implementing example embodiments. Further example embodiments may include a non-transitory computer-readable medium containing instructions that may be executed by a processor, and, when loaded and executed, cause the processor to complete methods described herein. It should be understood that elements of the block and flow diagrams may be implemented in software or hardware, such as via one or more arrangements of circuitry of FIG. 15, disclosed above, or equivalents thereof, firmware, a combination thereof, or other similar implementation determined in the future.

In addition, the elements of the block and flow diagrams described herein may be combined or divided in any manner in software, hardware, or firmware. If implemented in software, the software may be written in any language that can support the example embodiments disclosed herein. The software may be stored in any form of computer readable medium, such as random access memory (RAM), read only memory (ROM), compact disk read-only memory (CD-ROM), and so forth. In operation, a general purpose or application-specific processor or processing core loads and executes software in a manner well understood in the art. It should be understood further that the block and flow diagrams may include more or fewer elements, be arranged or oriented differently, or be represented differently. It should be understood that implementation may dictate the block, flow, and/or network diagrams and the number of block and flow diagrams illustrating the execution of embodiments disclosed herein.

The teachings of all patents, published applications and references cited herein are incorporated by reference in their entirety.

While example embodiments have been particularly shown and described, it will be understood by those skilled in the art that various changes in form and details may be made therein without departing from the scope of the embodiments encompassed by the appended claims.

What is claimed is:

1. A computer-implemented method for providing orthodontic treatment options for use in orthodontic diagnosis or treatment planning, the method comprising:
performing a rules-based expert system analysis on a given feature variable to produce expert system treatment options, the given feature variable representing an orthodontic feature of a patient, the expert system treatment options being fewer in number than a number of standard orthodontic treatment options that apply to orthodontic diagnosis, orthodontic treatment options, or both;
applying a computer-implemented multi-component model to a given set of feature variables to produce multi-component model-based treatment options that include primary and secondary model-based treatment options; and
comparing the expert system treatment options to the multi-component model-based treatment options to determine disagreement or agreement between each other, wherein:
if disagreement, enabling an expert to review the expert system treatment options and the multi-component model-based treatment options, and adapting at least one of the given feature variable, rules-based expert system analysis, or multi-component model based on feedback from the expert; and
if agreement, outputting the primary and secondary model-based treatment options to a clinician.

2. The computer-implemented method of claim 1, further comprising providing text-based information to the clinician with the primary and secondary model-based treatment options, the text-based information relating to an interpretation produced by the multi-component model.

3. The computer-implemented method of claim 1, wherein the multi-component model includes at least two computer-implemented methods that produce respective results having a characteristic of at least one of interpretability, reliability, or accuracy, and wherein a result with at least one of each characteristic is produced by the multi-component model.

4. The computer-implemented method of claim 3, wherein the multi-component model performs:
a multi-class logistic regression that produces a reliable result, interpretable result for a specific treatment option, or both, the specific treatment option including a location or identity of tooth extraction or other multi-class diagnosis or treatment;
a logistic regression that produces an interpretable and reliable result for an extraction option, non-extraction option, or other binary decision for diagnosis or treatment; and
a random forest method that produces an accurate result based on previous expert-decisions based training.

5. The computer-implemented method of claim 4, wherein the multi-class logistic regression is performed by a neural network including 0 or more hidden layers.

6. The computer-implemented method of claim 4, wherein:
the logistic regression, multi-class logistic regression, or combination thereof, is replaced by a decision tree, linear regression, generalized linear model, decision rules, RuleFit, naïve Bayes, k nearest neighbors, or one or more other interpretable machine learning method; and
the random forest is replaced by one or more of other machine learning methods with learning capacity and generalizability, which may or may not have interpretability, the one or more other machine learning methods including deep neural networks or other ensemble methods, the other ensemble methods including XGBoost, bagging, boosting, support vector machines, or a combination thereof.

7. The computer-implemented method of claim 4, further comprising integrating goals of interpretability, reliability, and accuracy using one integrated machine learning method that fuses the ideas or components of other methods.

8. The computer-implemented method of claim 1, wherein the given feature variable is a set of feature variables of a patient's orthodontia discernible from at least one of an x-ray, picture, physical model of the patient's orthodontia, or combination thereof, and wherein a number of feature variables in the set is within a range of: 1-7, 1-70, or 1-700.

9. The computer-implemented method of claim 8, further comprising performing automatic or user assisted feature identification on the x-ray, picture, model, or combination thereof, to produce the set of feature variables.

10. The computer-implemented method of claim 1, wherein the given feature variable is a set of feature variables and wherein the method further comprises (i) performing a corresponding rules-based expert system analysis on each feature variable of the set, (ii) applying the multi-component model to each feature variable, and (iii) performing the comparing, enabling, and outputting based on results of (i) and (ii).

11. The computer-implemented method of claim 1, wherein the expert is an expert clinician, expert panel of clinicians, or computer-implemented artificial intelligence or adaptive learning system.

12. The computer-implemented method of claim 1, further comprising performing a safety check of the rules-based expert system analysis based on a result of the computer-implemented multi-component model and replacing the safety check by other well-accepted orthodontic standards.

13. The computer-implemented method of claim 1, wherein features used for the rules-based expert system analysis or computer-implemented multi-component model are qualitative and categorical variables that are easily understood and used in the clinical setting.

14. The computer implemented method of claim 1, further comprising enabling a user to interact with a central server implementing the computer-implemented multi-component model through use of a visual or text based interface on a computer, phone, tablet, or other electronic device.

15. The computer implemented method of claim 1, further comprising enabling a user to select an option to store patient data of the patient either on selected equipment or on a central server.

16. The computer-implemented method of claim 1, further comprising automatically deriving at least one orthodontic feature of the patient from patient X-rays or other images by human intervention.

17. The computer-implemented method of claim 1, wherein the method further comprises recommending or ruling out braces, aligners, tooth extraction, or other diagnoses or treatments.

18. A system for providing orthodontic treatment options for use in orthodontic diagnosis or treatment planning, the system comprising at least one processor configured to:
perform a rules-based expert system analysis on a given feature variable to produce expert system treatment options, the given feature variable representing an orthodontic feature of a patient, the expert system treatment options being fewer in number than a number of standard orthodontic treatment options that apply to orthodontic diagnosis, orthodontic treatment options, or both;
apply a computer-implemented multi-component model to a given set of feature variables to produce multi-component model-based treatment options that include primary and secondary model-based treatment options; and
compare the expert system treatment options to the multi-component model-based treatment options to determine disagreement or agreement between each other, wherein:
if disagreement, the at least one processor is further configured to enable an expert to review the expert system treatment options and the multi-component model-based treatment options, and adapting at least one of the given feature variable, rules-based expert system analysis, or multi-component model based on feedback from the expert; and
if agreement, the at least one processor is further configured to output the primary and secondary model-based treatment options to a clinician.

19. The system of claim 1, wherein the system is integrated into an electronic medical records system.

20. A non-transitory computer-readable medium for providing orthodontic treatment options for use in orthodontic diagnosis or treatment planning, the non-transitory computer-readable medium having encoded thereon a sequence of instructions which, when loaded and executed by at least one processor, causes the at least one processor to:
perform a rules-based expert system analysis on a given feature variable to produce expert system treatment options, the given feature variable representing an orthodontic feature of a patient, the expert system treatment options being fewer in number than a number of standard orthodontic treatment options that apply to orthodontic diagnosis, orthodontic treatment options, or both;
apply a computer-implemented multi-component model to a given set of feature variables to produce multi-component model-based treatment options that include primary and secondary model-based treatment options; and
compare the expert system treatment options to the multi-component model-based treatment options to determine disagreement or agreement between each other, wherein:
if disagreement, the sequence of instructions further causes the at least one processor to enable an expert to review the expert system treatment options and the multi-component model-based treatment options, and adapt at least one of the given feature variable, rules-based expert system analysis, or multi-component model based on feedback from the expert; and
if agreement, the sequence of instructions further causes the at least one processor to output the primary and secondary model-based treatment options to a clinician.
